(19) United States
(12) Patent Application Publication
(10) Pub. No.: US 2015/0157486 A1
(43) Pub. Date: Jun. 11, 2015

(54) SUPPORT DEVICE

(71) Applicant: Walter J. Wolanske, Buffalo, NY (US)

(72) Inventor: Walter J. Wolanske, Buffalo, NY (US)

(21) Appl. No.: 14/100,693

(22) Filed: Dec. 9, 2013

(51) Int. Cl. A61F 5/02

(52) U.S. Cl. CPC A61F 5/028

(57) ABSTRACT

A support device (20) includes: a band (21) adapted to encircle a portion of the person's body. The band has a rear portion (22), side portions (23, 24) extending away from the rear portion in opposite directions, and marginal end portions (25, 26) that are adapted to overlap one another. At least one tightening mechanism (28) is mounted on the band. Each tightening mechanism includes: a pair of eyelets (29, 29) secured to the band, a single trace (30) having its marginal end portions secured to the band and having portions passing through the eyelets, a pull-strip (31) adapted to be secured to the band, and having another marginal end portion (33), and an eyelet (34) secured to such pull-strip other marginal end portion. An intermediate portion of the trace is passed through the pull-strip eyelet. The pull-strip may be used to selectively tighten the band about the portion of the person's body.

### TECHNICAL FIELD

The present invention relates generally to the field of support devices, such as back braces and lumbar orthotic devices, and, more particularly, to improved back braces that can be quickly and easily tightened, and that can automatically equalize the forces exerted on the upper and lower portions of the brace independently of various movements of the wearer's torso.

### BACKGROUND ART

Many people suffer from back pain.

There are many possible causes of this pain. In some cases, the pain may be reduced or alleviated by a suitable brace or support. Back braces are certainly known.

For example, one prior art brace is shown and described in U.S. Pat. No. 7,001,348 B2. This reference discloses an adjustable back brace with a rear portion, two side portions, and overlapable marginal end portions. Two pull-tabs are adjustably mounted on each of the side portions, and may be used to selectively tighten upper and lower cords at the rear of the brace. The pull-tabs may be operated separately to adjust the cords independently of one another. Thus, it is possible for the upper portion of the brace to be tightened more than the lower portion, or vice versa, and this situation may be further complicated when the wearer stands or sits.

Another prior art back brace is shown and described in U.S. Pat. No. 7,449,006 B2. This patent discloses an equalizing lumbar orthosis having a rear portion, two side portions, and overlapable marginal end portions. Two traces have first marginal end portions secured to the side portions, have intermediate portions passing through eyelets secured to the rear portion, and have distal marginal end portions secured to an intermediately-pivoted member that somewhat resembles a whiffle-tree. Pull-tabs are adjustably mounted on the side portions, and may be used to selectively pull the intermediately-pivoted member to tighten the traces. However, when the pull-tabs are adjusted, the traces may not be tightened evenly. For example, if the wearer is shaped like an apple, the upper trace may be tightened more than the lower trace when the pull-tab is adjusted. Moreover, when the wearer's torso moves, the intermediately-pivoted member may cock or tilt such that unequal forces will be exerted on the traces.

Other details of prior art braces are shown and described in U.S. Pat. No. 6,666,838 B2, U.S. Pat. No. 6,610,022 B1, U.S. Pat. No. 6,213,968 B1, and U.S. Pat. No. 6,964,644 B1.

The aggregate disclosures of each of the patents cited in this section are hereby incorporated by reference.

### DISCLOSURE OF THE INVENTION

With parenthetical reference to the corresponding parts, portions or surfaces of the disclosed embodiment, merely for purposes of illustration, and not by way of limitation, the present invention broadly provides an improved support device (20) that is adapted to be worn by a person (not shown).

The improved support device includes: a band (21) adapted to encircle a portion of the person's body, the band having a rear portion (22) adapted to be positioned proximate the rear of the person's body portion, having side portions (23, 24) extending away from the rear portion in opposite directions, and having marginal end portions (25, 26) that are adapted to overlap one another proximate the front of the person's body portion, the marginal end portions being adapted to be selectively secured to one another such that the band may encircle the portion of the person's body; and at least one tightening mechanism (28) mounted on the band, the tightening mechanism including: a pair of eyelets (29, 29) secured to the band, a single trace (30) having its marginal end portions secured to the band and having portions passing through the eyelets, a pull-strip (31) having one marginal end portion (32) adapted to be secured to the band at any of a plurality of positions relative thereto, and having another marginal end portion (33), and an eyelet (34) secured to such pull-strip other marginal end portion, and wherein an intermediate portion of the trace is passed through the pull-strip eyelet, whereby the pull-strip may be grasped, pulled away from the rear portion and secured to the band to selectively tighten the band about the portion of the person's body, and the tightening mechanism may self-adjust to accommodate various movements of the person's body portion.

As used herein, references to the “rear” and “front” of a person's body portion are intended to be relative, as opposed to absolute, terms. In other words, the “front” of a person's body portion is meant to be generally opposite to the “rear” of the person's body portion. The terms are not intended to have absolute directional significance, i.e., “rear” does not invariably mean facing rearwardly, and “front” does not invariably mean facing forwardly.

The trace marginal end portions may be secured to the band proximate the band rear portion.

The pair of eyelets may be secured to the band at spaced locations thereon.

The band may be adapted to encircle a portion of the person's torso.

The band may be adapted to encircle a lower portion of the person's torso so as to provide support for the person's lumbar region.

Accordingly, the general object of the invention is to provide an support device. Another object is to provide an improved back brace that is automatically self-adjusting and force-equalizing in the sense that the upper and lower portions will be placed under equal tensile loads by the simple action of a pulling a pull-strip.

Still another object is to provide an improved support device that will self-adjust to exert substantially equal forces on the upper and lower portions independently of the position of the wearer's torso (i.e., whether standing, sitting or moving).

These and other objects and advantages will become apparent from the foregoing and ongoing written specification, the drawings, and the appended claims.

### BRIEF DESCRIPTION OF THE DRAWINGS

FIG. 1 is an elevation looking at the exterior surface of the improved back brace, in a folded-flat condition, this view showing the traces, and the left and right pull-strips.

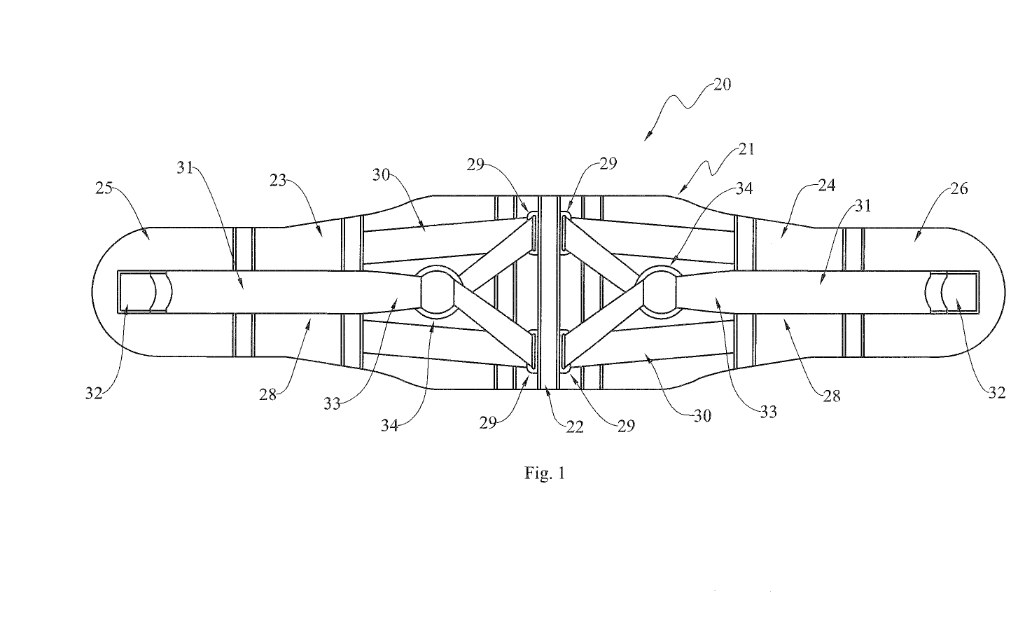

### DESCRIPTION OF THE PREFERRED EMBODIMENTS

At the outset, it should be clearly understood that like reference numerals are intended to identify the same structural elements, portions or surfaces consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate

Referring now to the drawing, the present invention provides an improved support device, generally indicated at 20, that is adapted to be worn by a person or patient. While the improved device will be described as a back brace or lumbar orthotic device, it should be clearly understood that the use of the improved device is not limited to this application. Indeed, the improved device could be used to support other portions of a person's body, such as a knee, elbow or the like, as well.

The improved device 20 is shown as including a band, generally indicated at 21, that is adapted to encircle a portion of the person's body. The band has a rear portion 22 adapted to be positioned proximate the rear of the person's body portion, side portions 23, 24 extending horizontally away from the rear portion in opposite directions, and marginal end portions 25, 26 at the distal ends of the side portions. These marginal end portions are adapted to overlap one another proximate the front of the person's body portion, and be secured to one another (e.g., by hook-and-loop fasteners, or the like) such that the band may encircle the portion of the person's body.

The improved device is also shown as having at least one tightening mechanism, generally indicated at 28, mounted on the band. In the illustrated form, two such tightening mechanisms are provided, each on either side of the rear portion.

Each tightening mechanism 28 includes a pair of vertically-spaced eyelets, severally indicated at 29, secured to the band proximate the rear portion 22 thereof, a single trace 30 having its marginal end portions secured to the band side portions, and having portions passed though eyelets 29, 29.

A pull-strip 31 has one marginal end portion 32 adapted to be selectively secured to the band at any of a plurality of positions relative thereto (e.g., by hook-and-loop fasteners, or the like), and having another marginal end portion 33. An eyelet 34 is secured to the pull-strip other marginal end portion. An intermediate portion of each trace is passed through the pull-strip eyelet.

The distal end portions of each pull-strip may be grasped, pulled away from the rear portion of the band and re-secured to the band to selectively tighten the band about the portion of the person's body.

The tightening mechanism may self-adjust to accommodate various movements of the person's body portion. Some people are built like apples. However, by having the pull-tab exert a force on the single trace via eyelet 34, the tightening mechanism may self-adjust regardless of whether the person is sitting, standing, or otherwise moving.

As previously noted, one object of the improved device is to function as a back brace or lumbar orthosis. In this application, the band is adapted to encircle a portion of the person's torso so as to provide support for the person's lumbar region. However, this is not the only application, and the improved device may be used to support other portions of the person's body as well.

Therefore, the present invention broadly provides an improved support device 20 that is adapted to be worn by a person. The improved support device broadly includes: a band 21 adapted to encircle a portion of the person's body, the band having a rear portion 22 adapted to be positioned proximate the rear of the person's body portion, having side portions 23, 24 extending away from the rear portion in opposite directions, and having marginal end portions 25, 26 that are adapted to overlap one another proximate the front of the person's body portion, the marginal end portions being adapted to be selectively secured to one another such that the band may encircle the portion of the person's body, and at least one tightening mechanism 28 mounted on the band, the tightening mechanism including: a pair of eyelets 29, 29 secured to the band, a single trace 30 having its marginal end portions secured to the band and having portions passing through the eyelets, a pull-strip 31 having one marginal end portion 32 adapted to be secured to the band at any of a plurality of positions relative thereto, and having another marginal end portion 33, and an eyelet 34 secured to such pull-strip other marginal end portion, and wherein an intermediate portion of the trace is passed through the pull-strip eyelet. The pull-strip may be grasped, pulled away from the rear portion and secured to the band to selectively tighten the band about the portion of the patient's body, and the tightening mechanism may self-adjust to accommodate various movements of the person's body portion.

### Modifications

The present invention expressly contemplates that many changes and modifications may be made.

For example, the device may be formed of suitable fabrics and other materials. The eyelets may have the shape and form shown, or may have other shapes and forms. The pull-tab may be secured to the band by means of hook-and-loop fasteners, or by some other means as well.

As used herein, the word “person” is used to indicate a patient, whether human or not.

Therefore, while the presently-preferred form of the improved support device has been shown and described, and some changes and modifications discussed, persons skilled in this art will readily appreciate that various additional changes and modifications may be made without departing from the spirit of the invention, as defined and differentiated by the following claims.

What is claimed is:

1. A support device adapted to be worn by a person, comprising:
a band adapted to encircle a portion of said person's body, said band having a rear portion adapted to be positioned proximate the rear of said person's body portion, having side portions extending away from said rear portion in opposite directions, and having marginal end portions that are adapted to overlap one another proximate the front of said person's body portion, said marginal end portions being adapted to be selectively secured to one another such that said band may encircle said portion of said person's body; and
at least one tightening mechanism mounted on said band, said tightening mechanism including:
a pair of eyelets secured to said band,
a single trace having its marginal end portions secured to said band and having portions passing through said eyelets,
a pull-strip having one marginal end portion adapted to be secured to said band at any of a plurality of positions relative thereto, and having another marginal end portion, and
an eyelet secured to such pull-strip other marginal end portion, and wherein an intermediate portion of said trace is passed through said pull-strip eyelet,
whereby said pull-strip may be grasped, pulled away from said rear portion and secured to said band to selectively tighten said band about said portion of said person's body, and said tightening mechanism may self-adjust to accommodate various movements of said person's body portion.

2. A support device as set forth in claim 1 wherein said trace marginal end portions are secured to said band proximate said band rear portion.

3. A support device as set forth in claim 1 wherein said pair of eyelets are secured to said band at spaced locations thereon.

4. A support device as set forth in claim 1 wherein said band is adapted to encircle a portion of said person's torso.

5. A support device as set forth in claim 4 wherein said band is adapted to encircle a lower portion of said person's torso so as to provide support for the person's lumbar region.
